(19) United States
(12) Patent Application Publication
(10) Pub. No.: US 2007/0185402 A1
(43) Pub. Date: Aug. 9, 2007

(54) Airbag having a stable pressure release device operated with a sphygmomanometer

(75) Inventors: Paul Yang, Taipei (TW); Shan-Yi Yu, Taipei (TW); Hsin-Yi Pai, Nantou County (TW); Shao-Hung Lee, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., Chung Ho City (JP)

(21) Appl. No.: 11/348,406

(22) Filed: Feb. 7, 2006

(51) Int. Cl. A61B 5/02

(52) U.S. Cl. 600/490

(57) ABSTRACT

An airbag having a stable pressure release device operated with a sphygmomanometer includes a casing having an interconnecting pipe with a solenoid valve for discharging air from an airbag and an air escape valve for discharging air at a low airflow, when measuring blood pressure. The airflow is adjusted for a standard sized airbag, and the interconnecting pipe is connected to the airbag. The airbag is larger than a standard sized airbag. The airbag has a porous piston. If a larger arm is measured, the air is discharged through the air escape valve with a low airflow, and the air holes of the piston can increase the airflow and stabilize the pressure release, so that a more accurate pulse signal can be obtained.

### BACKGROUND OF THE INVENTION

1. Field of the Invention

The present invention relates to an airbag having a stable pressure release device operated with a sphygmomanometer, and more particularly to an airbag that installs a piston made of a porous material into an airbag that is worn around a user's arm, such that sphygmomanometer manufacturers just need to use the same standard for manufacturing an air escape valve and discharging an airflow to control a sphygmomanometer, and tiny even air holes in the piston of the large airbag can increase the airflow for the large airbag, so as to achieve the effect of stabilizing the pressure release through the piston and the air escape valve in the sphygmomanometer.

2. Description of the Related Art

Referring to FIG. 1 for the prior art sphygmomanometer, the sphygmomanometer comprises an interconnecting pipe 100, and an end of the interconnecting pipe 100 is connected to an electric pump 110, and another end of the interconnecting pipe 100 is connected to an airbag 200 which is worn around an arm or a wrist of a user. Further, the airbag 200 is divided into a standard size and a large size to fit different arm sizes, wherein the standard size (fitting an arm with a diameter of 9 to 13 inches) is primarily designed for arms of a type of build of Orientals or general Europeans and Americans, and the large size (fitting an arm with a diameter of 13 to 17 inches) is primarily designed for arms of fat people.

Referring to FIGS. 1 and 1A, the interconnecting pipe 100 is connected separately to a pulse sensor 120, a solenoid valve 130, and an air escape valve 131, wherein the pulse sensor 120 is installed on the circuit board 140 for detecting the pulse of the blood pressure of an arm during a blood pressure measurement, and the high/low blood pressure is measured, and the circuit board 140 is connected to an LCD screen 150 through an electric circuit. Further, the solenoid valve 130 is provided for rapidly discharging the air in the airbag, and the air escape valve 131 is provided for discharging the air in the airbag with a low airflow during the process of measuring blood pressure. The high/low blood pressure is measured by the pulse sensor 120 after the airbag is inflated and pressurized to a predetermined maximum pressure, and the air in the airbag is steadily discharged with a low airflow in a fixed time. Therefore, the size of the airbag and the stability of releasing the pressure of airflow passing through the air escape valve 131 affect the accuracy of the measurement directly. The openness of the air escape opening 132 of the air escape valve 131 varies with the size of the airbag 200. For example, the large airbag 200 has a large capacity and thus takes a longer time for releasing the pressure during a blood pressure measurement. It is necessary to adjust and enlarge the size of the air escape opening 132 to speed up the airflow, and thus the openness of the air escape opening 132 of the sphygmomanometer must be adjusted according to the size of the connected airbag 200. However, the factory default standard of each sphygmomanometer is set to the standard sized airbag 200, such that if the sphygmomanometer is connected to a large airbag 200, the measured values will be inaccurate.

### SUMMARY OF THE INVENTION

In view of the shortcomings of the prior art, the inventor of the present invention aimed at the problem of the prior art sphygmomanometer that requires adjusting the openness of the air escape opening in the air escape valve to fit the airbag size and control the airflow and tried to find a way of overcoming the shortcomings of the prior art and conducted extensive researches and experiments to find a feasible solution, and finally invented a stable pressure release device for electronic sphygmomanometer in accordance with the present invention.

Therefore, it is an objective of the present invention to provide an airbag having a stable pressure release device operated with a sphygmomanometer that includes a casing, and the casing comprises an interconnecting pipe, a pressurizing device disposed at an end of the interconnecting pipe, and another end of the interconnecting pipe is connected to an airbag that is worn around an arm or a wrist of a user. The airbag is larger than the standard size and fits the large airbag worn on a large arm, and the airbag includes a piston made of a porous material, and the interconnecting pipe includes an air escape valve having an air escape opening, and the openness of the air escape opening is adjusted according to the airflow required for releasing the pressure of the standard sized airbag. If the present invention is used for a large airbag that fits a large arm, the air of the pressurizing device will be passed through the interconnecting pipe to inflate the airbag to a predetermined pressure. When blood pressure is measured, the pressure can be released from the air escape opening of the air escape valve as well as from the tiny even air holes in the piston of the large airbag to increase the airflow and stabilize the pressure release to the outside. Therefore, the pulse sensor can obtain a more accurate pulse signal of the blood pressure due to the steady airflow. The sphygmomanometer no longer needs to adjust the airflow of the air escape opening of the air escape valve, and such sphygmomanometer is applicable for both standard sized and large airbags, so as to save the time and cost of adjusting the air escape opening manually.

Another objective of the present invention is to provide an airbag having a stable pressure release device operated with a sphygmomanometer that includes a piston made of a precision ceramic material.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a schematic view of a prior art device;

FIG. 1A is an enlarged view of an air escape valve of a prior art device;

FIG. 2 is a schematic view of an airbag of the present invention;

FIG. 2A is an enlarged view of an airbag installed with a piston according to the present invention;

FIG. 3 is a schematic view of using an airbag according to the present invention;

FIG. 4 is a schematic view of a preferred embodiment of the present invention; and

FIG. 4A is an enlarged view of an air escape valve of a preferred embodiment of the present invention.

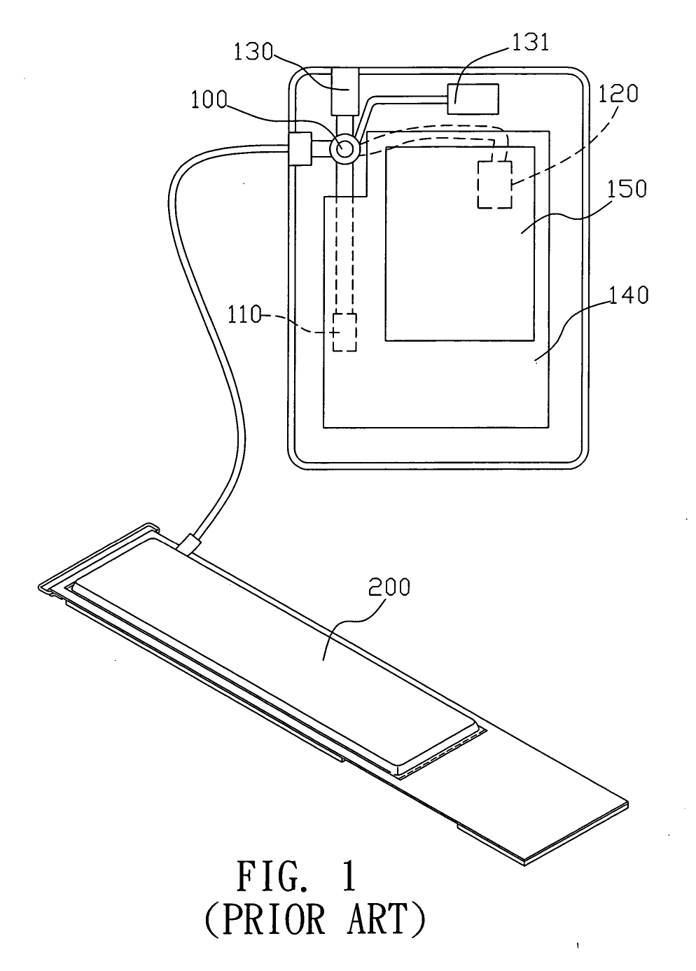

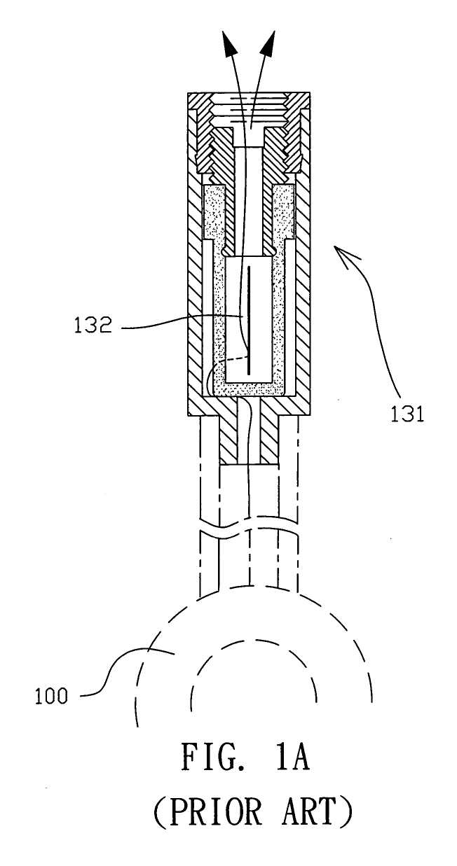

### DETAILED DESCRIPTION OF THE PREFERRED EMBODIMENTS

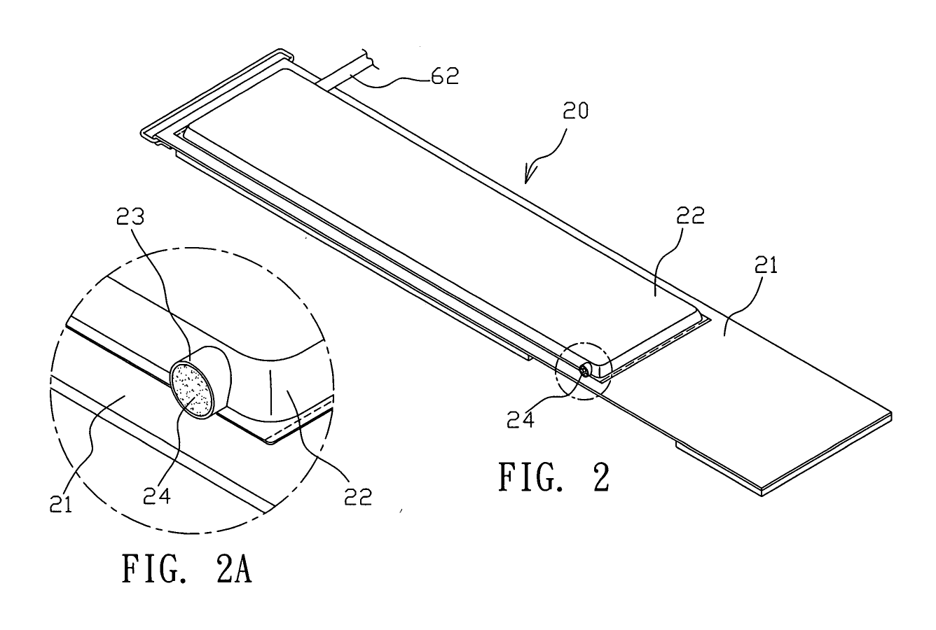

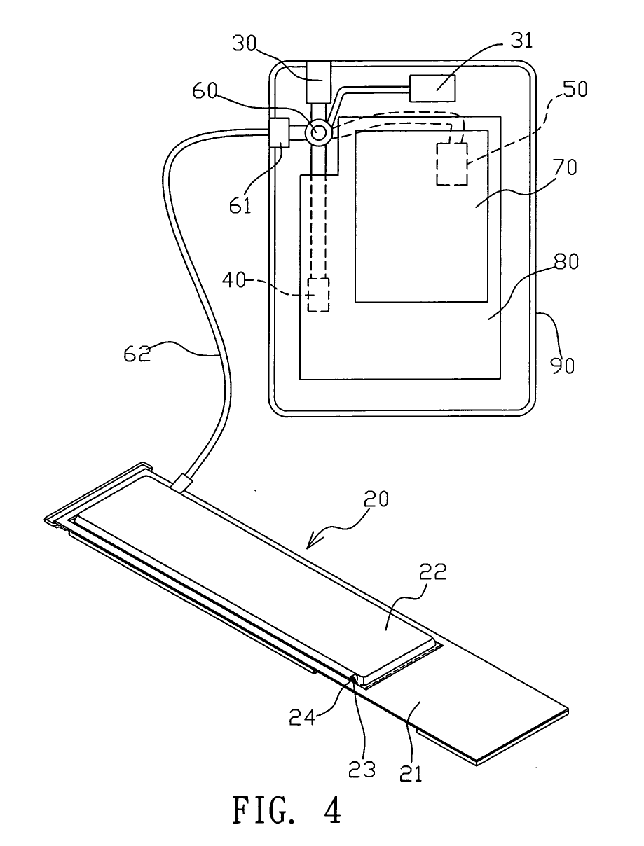

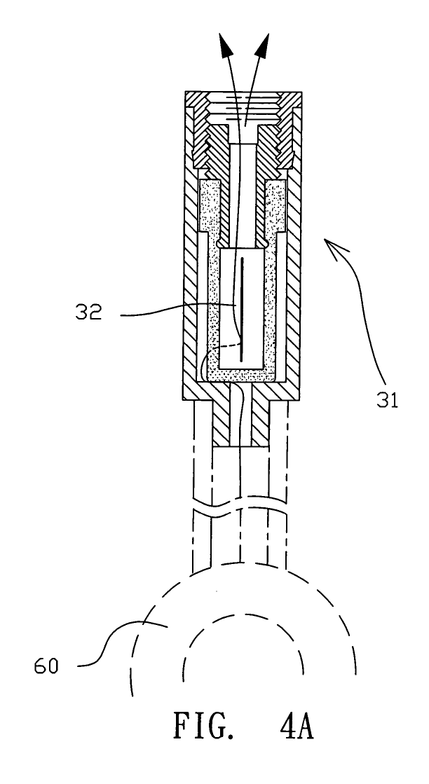

Referring to FIGS. 2 and 4 for the large airbag device used in a sphygmomanometer, the sphygmomanometer includes a casing 90, and the casing 90 contains a circuit board 80, and the circuit board 80 is connected to an LCD screen 70 which is exposed from the surface of the casing 90 for displaying the reading of a measured high/low blood pressure, and the casing 90 includes a multi-channel interconnecting pipe 60, and the interconnecting pipe 60 is connected to a connector 61, and the connector 61 is connected to an airbag 20 through a pipeline 62. In this preferred embodiment, a large airbag (fitting an arm with a diameter of 13 to 17 inches) is adopted, and the airbag 20 includes a strap 21 for wrapping around a user's arm, and the strap 21 holds an inflating bag 22, and the inflating bag 22 includes an opening 23 on one side, and a piston 24 made of a porous material is disposed in the opening 23 (as shown in FIGS. 2 and 2A). In this preferred embodiment, the piston 24 is made of precision ceramic, but the persons skilled in the art can use a foam material, a nano material, or similar substitutes as well.

Further, the interconnecting pipe 60 is connected separately to a pulse sensor 50 and a pressurizing device 40, wherein the pulse sensor 50 is installed on the circuit board 80 for detecting the pulse of blood pressure during a measurement of measuring the high/low blood pressure, and the pressurizing device 40 in this preferred embodiment is an electric pump, but the persons skilled in the art can use a manual pressurization ball to substitute the electric pump. The interconnecting pipe 60 is connected to a solenoid valve 30 and an air escape valve 31, and the openness of the air escape opening 32 of the air escape valve 31 (as shown in FIG. 4A) can be adjusted according to the airflow required for releasing the pressure of a standard sized airbag.

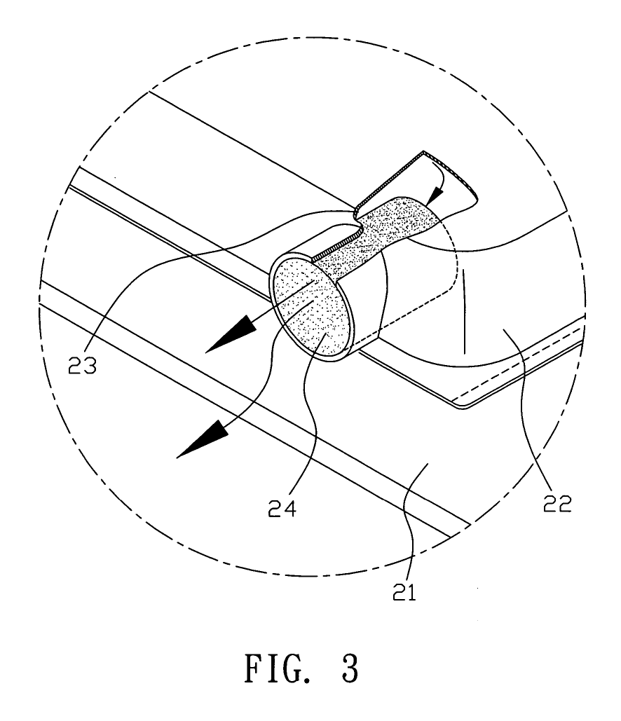

Referring to FIGS. 2 to 4, the airbag 20 is pressurized by sending the pressurized air from the pressurizing device 40 to the pipeline 62, such that the air passing through the pipeline 62 to a large airbag 20 worn around an arm or a wrist of a user. The pressurization will stop until the predetermined maximum, pressure is reached, so that when blood pressure is measured, the air in the large airbag 20 is discharged steadily to the outside through an air escape opening 32 of an air escape valve 31 in the sphygmomanometer by an airflow pressure release configured for the standard sized airbag and through the tiny even air holes in a piston 24 of the large airbag 20, so that the large airbag 20 and the standard sized airbag can simultaneously release pressure to a predetermined minimum air pressure, and the pulse sensor 50 can accurately detect the pulse signal of the blood pressure due to the tiny steady airflow. Since the piston 24 will not be exhausted easily, errors caused by an unstable flow will not occur. The pulse sensor 50 detects the pulse of the high/low blood pressure while the air is discharged slowly, and the detected high/low blood pressure pulse information is sent to the circuit board 80 for processing, and the measured result is sent to the LCD screen 70 for its display. Further, the present invention can be connected to a standard sized airbag for its use, such that the sphygmomanometer of the invention can be used for a standard sized airbag as well as a large airbag, and thus saving the time and cost for manual adjustments.

In summation of the above description, the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. An airbag having a stable pressure release device operated with a sphygmomanometer, and said sphygmomanometer having an interconnecting pipe, and said interconnecting pipe having a pressurizing device and an air escape valve, and the pressure release of an airflow through an air escape opening of said air escape valve being set according to a standard sized airbag, characterized in that: another end of said channel is coupled to a large airbag worn around an arm or a wrist of a user, and said large airbag includes a piston made of a porous material, such that when said sphygmomanometer is used, said pressurizing device pressurizes air and sends the air to said interconnecting pipe to pressurize said large airbag to a predetermined maximum pressure, and when a blood pressure is measured, the air in said airbag passes through said air escape opening of said air escape valve and tiny and even air holes in a piston of said large airbag and through air holes to the outside in a tiny quantity and a steady manner, without the need of adjusting the openness of said air escape opening of said air escape valve to increase the discharged airflow of said large airbag.

2. The airbag operated with a sphygmomanometer of claim 1, wherein said piston is made of a precision ceramic material.

3. The airbag operated with a sphygmomanometer of claim 1, wherein said pressurizing device is an electric pump.

4. The airbag operated with a sphygmomanometer of claim 1, wherein said pressurizing device is a manual pressurization ball.

5. The airbag operated with a sphygmomanometer of claim 1, wherein said large airbag is applicable for an arm with a diameter of 13˜17 inches.
